(19) United States
(12) Patent Application Publication
(10) Pub. No.: US 2022/0079894 A1
(43) Pub. Date: Mar. 17, 2022

(54) STABLE PHARMACEUTICAL COMPOSITIONS

(71) Applicant: Marksans Pharma Ltd., Mumbai (IN)

(72) Inventors: Mark Saldanha, Oshiwara (IN); Varddhman Vikramaditya Jain, Verna (IN)

(21) Appl. No.: 17/081,291

(22) Filed: Oct. 27, 2020

(30) Foreign Application Priority Data
Sep. 11, 2020 (IN) 202021039399

(51) Int. Cl. A61K 31/155; A61K 47/38; A61K 47/02; A61K 47/12; A61J 1/03

(52) U.S. Cl. CPC A61K 31/155; A61K 47/38; A61J 1/03; A61K 47/12; A61K 47/02

(57) ABSTRACT

A stable pharmaceutical composition utilizing a desiccant to reduce the presence of NDMA while the composition is in storage and a method of reducing and/or eliminating same. The Angiotensin II receptor antagonist (ARBs) drugs, Gastroesophageal Reflux Diseases (GERD) drugs and Anti Diabetic Drug are prone to formation of Nitrosamine impurities. These Nitrosamine impurities specifically can be significantly reduced and controlled in a container closure system by using the moisture absorption or adsorption agent. The present invention incorporates the use of a desiccant as a moisture absorption agent when the pharmaceutical is being stored in a sealed container.

### CROSS REFERENCE TO RELATED APPLICATIONS

This application claims priority of an Indian patent application Application Serial No. IN202021039399 filed on Sep. 11, 2020, entitled STABLE PHARMACEUTICAL COMPOSITIONS, which application is assigned to the same assignee as this application and whose disclosure is incorporated by reference herein.

### BACKGROUND OF THE INVENTION

Pharmaceutical compositions are expected to be safe and in general have to comply with the requirements of the ICH and US FDA regulations. Amongst other quality attributes, the level of impurities present in the pharmaceutical composition should comply with the requirement outlined in the ICH, USP and the US FDA. Particularly, the presence of N-Nitrosamine impurities in pharmaceutical compositions is undesirable due to their carcinogenic nature.

N-Nitrosamines are undesired industrial and environmental pollutants with rising concerns due to their widespread observation in foods, soil, industrial workplace environments, and cosmetics as well as due to their high carcinogenic risks. US Environmental Protection Agency (U.S. EPA) has prescribed six kinds of nitrosamines as probable B2 carcinogenic compounds to humans. See Genotoxic and carcinogenic risk to humans of drug-nitrite interaction products, Giovanni Brambilla*, Antonietta Martelli, Mutation Research 635 (2007) 17-52, incorporated by reference herein in its entirety in which it states that the large majority of N-nitroso compounds (NOC) have been found to produce genotoxic effects and to cause tumor development in laboratory animals; four NOC have been classified by the International Agency for Research on Cancer (IARC) as probably and another 15 as possibly carcinogenic to humans. See also, N-Nitrosodimethylamine (NDMA) as a Drinking Water Contaminant: A Review, William A. Mitch, et al., ENVIRONMENTAL ENGINEERING SCIENCE Volume 20, Number 5, 2003, incorporated herein, and states that N-Nitrosodimethylamine (NDMA) is a member of a family of extremely potent carcinogens, the N-nitrosamines.

In a review entitled Update on genotoxicity and carcinogenicity testing of 472 marketed Pharmaceuticals. Giovanni Brambilla and, Antonietta Martelli, Mutation Research 681 (2009) 209-229, are discussed a number of pharmaceutical formulations with such issues. See Table 1 for metformin.

In Inhibitors of endogenous nitrosation, Mechanisms and implications in human cancer prevention by Helmut Bartsch, Hiroshi Ohshima and Brigitte Pignatelli, Mutation Research, 202 (1988) 307-324, discussing that although the proof that N-nitroso compounds (NOC), a versatile class of carcinogens in animals, are also carcinogenic in man is lacking (as of 1988), humans are exposed through ingestion or inhalation to preformed NOC in the environment and through the endogenous nitrosation of amino precursors in the body. Activated macrophages can synthesize nitrate, nitrite and nitrosating agents that can form NOC.

Recent guidance from the US FDA https://www.fda.gov/drugs/drug-safety-and-availability/questions-and-answers-ndma-impurities-metformin-products has recommended recalls of certain metformin products that may contain the impurity N-nitrosodimethylamine (NDMA) above the acceptable intake limit. The agency is also asking all manufacturers of extended release versions of metformin to evaluate their risk of excessive NDMA and to test at-risk product before each batch is released onto the U.S. market. If testing shows NDMA above the acceptable intake limit, the manufacturer must inform the agency and should not release the batch to the U.S. market. The guidance further states that FDA's testing has shown elevated levels of NDMA in some extended release (ER) metformin formulations but not in the immediate release (IR) formulation or in the active pharmaceutical ingredient. The agency is also asking all manufacturers of extended release versions of metformin to evaluate their risk of excessive NDMA and to test at-risk product before each batch is released onto the U.S. market. If testing shows NDMA above the acceptable intake limit, the manufacturer must inform the agency and should not release the batch to the U.S. market. See also the FDA update on this topic dated Aug. 21, 1920 https://www.fda.gov/drugs/drug-safety-and-availability/fda-updates-and-press-announcements-ndma-metformin

N-Nitrosodimethylamine (NDMA), is the simplest molecule in structure among all the nitrosamines and has been demonstrated to be one of the most carcinogenic, mutagenic, and teratogenic nitrosamines. In the U.S. EPA Integrated Risk Information Service (IRIS) database, NDMA has been identified to have an estimated 10⁻⁶ lifetime cancer risk level at a concentration as low as 0.7 ng/L. Although no national regulation controlling NDMA has been established yet, the Ontario Ministry of the Environment and the California Department of Public Health have a maximum allowable concentration (MAC) of 9 and 10 ng/L respectively.

The FDA has been investigating the presence of impurities, called nitrosamines, in some types of medications. Nitrosamines are common in water and foods, including cured and grilled meats, dairy products and vegetables. Everyone is exposed to some level of nitrosamines. The FDA, in collaboration with regulatory counterparts around the world, has set internationally-recognized acceptable daily intake limits for nitrosamines. Nitrosamine impurities may increase the risk of cancer if people are exposed to them above acceptable levels and over long periods of time, but a person taking a drug that contains nitrosamines at-or-below the acceptable daily intake limits every day for 70 years is not expected to have an increased risk of cancer.

There are multiple reasons why nitrosamines can be present in drugs. It has been found that the source of nitrosamines can be related to the drug's manufacturing process or its chemical structure or even the conditions in which they are stored or packaged. As foods and drugs are processed in the body, nitrosamines can also be formed.

Of particular concern is the level of NDMA in a class of anti-diabetic drugs including metformin hydrochloride tablets. Since these drugs are administered over a long period of time to patients, the amount of NDMA impurity in such products is of particular concern. It has been established that the 96 ng is the Acceptable Daily Intake Limit (ADI) for N-Nitrosodimethylamine (NDMA) in Metformin Hydrochloride tablets. The levels of NDMA in metformin hydrochloride tablets can be determined by analytical methods like Liquid Chromatography-Mass Spectrometry. Other drugs that can have similar issues are anti-diabetic drugs, a drug to treat Gastroesophageal Reflux Diseases (GERD) or an Angiotensis II receptor antagonist drug (ARBs).

Metformin Hydrochloride is a biguanide indicated as an adjunct to diet and exercise to improve glycemic control in adults with type 2 diabetes mellitus. It is available amongst other dosage forms as an immediate release tablet as well as an extended release tablet. The extended release tablet has advantages for the patients due to lesser frequency of taking the tablet in a day. The tablets are generally supplied by packing them in plastic bottles. For e.g., HDPE (High Density Polyethylene) bottles are used for packing Metformin Extended Release Tablets.

See for example, NDMA impurities in valsartan and other pharmaceutical products: Analytical methods for the determination of N-nitrosamines, Parr et al., Journal of Pharmaceutical and Biomedical Analysis, 164 (2019) pp. 536-549, the entire disclosure of which is incorporated by reference herein.

Metformin Extended Release Tablets 500 mg when tested by a LC-MS method for NDMA showed that the content of NDMA was 0.08 ug/gm. This meant that the potential daily exposure of NDMA in the product was 160 ng/day

| | | | NDMA Maximum |
|---|---|---|---|
| | | NDMA content | Daily exposure |
| Product | Lot No | ug/gm | ng/day |
| Metformin | XP9004 | 0.08 | 160 |
| Hydrochloride | | | |
| Extended release | | | |
| tablets 500 mg | | | |

There was no detectable NDMA in the active substance used in the above lot. Thus the NDMA appeared to be formed in the tablet composition.

### SUMMARY OF THE INVENTION

It is necessary to reduce the amount of NDMA formed in a pharmaceutical tablet so as to meet safety standards and reduce the risk of NDMA exposure to patients taking Angiotensis II receptor antagonist (ARBs) drugs, Gastroesophageal Reflux Diseases (GERD) drugs and antidiabetic drug like Metformin extended release tablets and other anti-diabetic drugs. The present invention incorporates the use of a desiccant as a moisture absorption agent when the pharmaceutical is being stored in a sealed container.

### DETAILED DESCRIPTION OF THE INVENTION

Metformin Hydrochloride extended release tablets are prone to formation of Nitrosamine Impurities as are other drugs. These Nitrosamine Impurities specifically can be significantly reduced and controlled in a container closure system by using the moisture absorption or adsorption agent which may include but not limited to a Silica base desiccant, Activated Charcoal or equivalents or a combination approach. During this finding, the amount of NDMA formed in Metformin Hydrochloride extended release tablets by adding a moisture scavenger in the container closure system has reduced and controlled the formation of the N-Nitrosodimethylamine (NDMA) impurity when compared with system containing only Metformin Hydrochloride extended release tablets.

The invention to inhibit the formation of Nitrosodimethylamine (NDMA) impurity can be extended to the other class of drugs like Gastroesophageal Reflux Diseases (GERD) drugs e.g., Nizatidine, ranitidine and Angiotensis II receptor antagonists (ARBs) like Lorasrtan, Telemisartan and Irbesartan which are prone to formation of an NDMA impurity during storage.

### Specific Embodiments of the Invention

A laboratory batch of Metformin Hydrochloride extended release tablets 750 mg was manufactured.

Batch No SD (167) 65- Metformin Hydrochloride
Extended Release Tablets 750 mg

| S.No | Ingredient | Mg/Tab |
|---|---|---|
| 1 | Metformin HCl USP | 750.00 |
| 2 | Hypromellose USP K 100 M CR | 405.00 |
| 3 | Colloidal Sillicon Dioxide | 8.00 |
| 4 | Magnesium Stearate | 12.00 |

The tablets are then packed into HDPE bottles fitted with PP screw caps. One part of the batch was packed in bottles with desiccant while another part was packed in bottles without desiccant. The bottles were then stored at 40° C./75% RH and the NDMA content was determined by a suitable method at different time intervals.

| Sr. | Sample | | Description of container | |
|---|---|---|---|---|
| No. | identification | Pack | closure system | Tests done |
| 1 | SD (167)65D | HDPE bottles | 20's count in 40 cc HDPE | NDMA content - Initial, |
| | | | bottle | 4 weeks and 8 weeks at |
| | | | | 40° C./75% RH |
| 2 | SD (167)65D | HDPE bottles | 20's count in 40 cc HDPE | NDMA content - Initial, |
| | | with silica gel | bottle and 2 gm silica gel | 4 weeks and 8 weeks at |
| | | dessicant | dessicant sachet | 40° C./75% RH |

It was surprisingly discovered that the packs containing the desiccant did not show any detectable amount of NDMA after 8 weeks at 40° C./75% RH, whereas the packs without desiccant showed NDMA formation.

### Results

| | | Initial | 40° C./75% RH | 40° C./75% RH |
|---|---|---|---|---|
| | | NDMA | 4 week | 8 week |
| | | content | NDMA content | NDMA content |
| | | (ppm) | (ppm) | (ppm) |
| Batch No. | Description | Initial | 4 Weeks | 8 Weeks |
| SD (167)065D | Tablets in bottles | ND | 0.010 | 0.034 |
| | (no dessicant) | | | |
| SD (167)065D | Tablets in bottles | ND | ND | ND |
| | with dessicant | | | |

ND—Not Detected

These results were entirely unexpected, and the protective action of the desiccant could be due to its ability to prevent the formation of a surface acidic environment in the product/environment which can slow down the nitrosation and associated reaction to form NDMA.

While the invention has been described in detail and with reference to specific examples thereof, it will be apparent to one skilled in the art that various changes and modifications can be made therein without departing from the spirit and scope thereof.

What is claimed is:

1. A method of packaging a pharmaceutical formulation comprising storing of:
a) at least one pharmaceutical formulation selected from an anti-diabetic drug, a Gastroesophageal Reflux Diseases (GERD) drug or an Angiotensis II receptor antagonist (ARBs) and
b) a desiccant;
in a sealed container to prevent the formation and/or reduction of N-nitrosodimethylamine (NDMA) as an impurity.

2. The formulation of claim 1 wherein the desiccant is selected from the group consisting of a silica-based desiccant, activated charcoal and combinations thereof and is contained within a container to prevent and/or reduce interaction with the pharmaceutical formulation.

3. The formulation of claim 1 wherein the desiccant prevents the formation of a surface acidic environment on the pharmaceutical formulation to reduce nitrosation and associated reactions to form NDMA thereon.

4. The formulation as claimed in any of the claim 1 wherein the pharmaceutical formulation is selected from group consisting of an anti-diabetic drug, a Gastroesophageal Reflux Diseases (GERD) drug or an Angiotensis II receptor antagonist (ARBs).

5. The formulation as claimed in claim 2 wherein the pharmaceutical formulation is selected from group consisting of an anti-diabetic drug, a Gastroesophageal Reflux Diseases (GERD) drug or an Angiotensis II receptor antagonist (ARBs).

6. The formulation as claimed in claim 3 wherein the pharmaceutical formulation is selected from group consisting of an anti-diabetic drug, a Gastroesophageal Reflux Diseases (GERD) drug or Angiotensis II receptor antagonist (ARBs).

7. The formulation as claimed in claim 1 wherein the anti-diabetic drug is metformin hydrochloride.

8. The formulation as claimed in claim 1 wherein the Gastroesophageal Reflux Diseases (GERD) drug is Ranitidine or Nizatidine.

9. The formulation as claimed in claim 1 wherein the Angiotensin II receptor antagonist (ARBs) is selected from the group consisting of Losartan, Valsartan and Irbesartan.

10. The formulation of claim 1 stored in a sealed container comprised of high-density polyethylene.
